(19) United States
(12) Patent Application Publication
(10) Pub. No.: US 2023/0201502 A1
(43) Pub. Date: Jun. 29, 2023

(54) Ventilator and Method for Determining at Least the Tissue-Related Resistance in the Respiratory Tract

(71) Applicant: Ventinova Technologies B.V., Eindhoven (NL)

(72) Inventors: Dietmar Enk, Coesfeld (DE); Thomas Heinrich Barnes, Warlingham Surrey (GB)

(21) Appl. No.: 17/927,129

(22) PCT Filed: May 17, 2021
(86) PCT No.: PCT/EP2021/062937

§ 371 Date: Nov. 22, 2022

(30) Foreign Application Priority Data
May 27, 2020 (DE) 10 2020 114 209.9

(51) Int. Cl. A61M 16/00; A61B 5/085

(52) U.S. Cl. CPC A61M 16/024; A61B 5/085; A61M 16/0003; A61M 2016/0027

(57) ABSTRACT

The invention relates to a ventilator (1) at least comprising: a gas supply device (2) and a gas discharge device (3), for supplying a first fluid flow (4) to a respiratory tract (5) of a patient and for discharging a second fluid flow (6) from the respiratory tract (5) back into the ventilator (1) or to a surrounding area (7); a pressure sensor (8) for sensing a pressure (9) in the respiratory tract (5); and a control device (10) for operating the ventilator (1); wherein the fluid flow (4, 6) can be set to a constant value at least during an inspiration process (11) and an expiration process (12). The invention further relates to a method for determining at least a tissue-related resistance of a patient by means of a ventilator (1).

The invention relates to a ventilator and a method for (differentiated) measurement or determination of indices or at least the tissue-related resistance in the airway of a patient, optionally additionally for measurement or determination of the airway-related resistance and for ascertainment of the (global) alveolar pressure or pressure plot (in the alveoli) of a patient.

Peak inspiratory pressure (PIP) refers to the highest positive pressure [in mbar, i.e., millibars] that is artificially generated in the airway during inhalation (inspiration).

End-inspiratory (plateau) pressure is the pressure that is measured in the airway at the end of inspiration.

End-expiratory (plateau) pressure, which is maintained in the airway after completion of exhalation (expiration), is preferably positive and is therefore also referred to as positive end-expiratory pressure (PEEP). In the following, reference is always made to PEEP.

Compliance [in ml/mbar, i.e., milliliters/millibar] is a measure of the expandability or compression resilience of the lung/thorax system of a patient. Under ventilation conditions, so-called static compliance is calculated by using tidal volume (VT) [in ml], which refers to the volume of air supplied during an inspiration, and the difference between end-inspiratory (plateau) pressure (e.g., Pₗ₁) and end-expiratory (plateau) pressure (e.g., P_{E1}).

By contrast, so-called dynamic compliance is calculated on the basis of V_{T} and the difference between PIP (e.g., Pₗ₂) and PEEP (e.g., PE2) [in mbar]. The pressure difference is therefore regularly greater in the case of dynamic compliance or at least as great as the pressure difference in the case of static compliance. Since compliance generally exhibits a changing relationship between pressure (P) and volume (V)—as pressure and volume change—it appears as a curve on a pressure-volume diagram.

Compliance thus indicates how much fluid (e.g., respiratory gas, i.e., a volume of air), i.e., a delta V, is introduced into the at least one airway or removed from the airway, such that a pressure in the airway changes by a pressure difference delta P. During at least one ventilation process (inspiration, i.e., the supply of fluid into the airway; expiration, i.e., the discharge of fluid from the airway), a plot of the compliance curve can be ascertained or additionally estimated (e.g., on the basis of empirical values). What can be ascertained in particular is the section of the compliance curve in which a certain volume (possibly V_{T}) can be supplied in a smallest possible pressure interval.

A user or preferably an (automatically operating) control device of a ventilator can thus, taking into account an ascertained or additionally estimated plot of at least a section of a compliance curve in a pressure-volume diagram, determine a position of a pressure interval having the pressures Pₗ and P_{E} and then set these pressures on the ventilator (e.g., PIP as Pₗ and PEEP as P_{E}), so that at least a ventilation process, i.e., an inspiration and/or an expiration, occurs between these pressures Pₗ and P_{E} and an absolute value of the compliance of this ventilation process is as large as possible.

Continuous ventilation should be set in such a way that a minute volume (i.e., V_{T} ventilation frequency [/min, i.e., ventilation processes per minute]) necessary for normoventilation (i.e., the adequate elimination or exhalation of carbon dioxide) is as small as possible and can be supplied and discharged at maximum possible compliance.

In contrast to static compliance, dynamic compliance must also take into account the resistances (in the broadest sense) to be overcome during inspiration or expiration, including effects of so-called ventilation history (i.e., how the lung was ventilated). The latter arises from the fact that the lung is a viscoelastic organ, the mechanical properties of which depend on how it is or was moved.

Resistance (measured in mbar/I/s; i.e., millibars/liter/second or mbar s/I, i.e., millibar second/liter) describes the resistances to be overcome during inspiration or expiration and indicates the pressure necessary for gas flow (fluid flow) and hence volume change (in the lung) per time.

In a ventilated patient, resistance is typically estimated during inspiration by measuring the pressure difference between peak inspiratory pressure (PIP) and end-inspiratory (plateau) pressure in relation to mean inspiratory flow (inspiratory fluid flow). The measurement requires a stop in the inspiratory flow.

For example, a pressure difference between peak inspiratory pressure (Pₗ₂) and end-inspiratory (plateau) pressure (PE2) of 2 mbar at a mean inspiratory flow of 18 I/min [liters/minute] yields an (inspiratory) resistance of 6.66 mbar/I/s; i.e., 2 mbar/18 I/min or 2 mbar·60 s/18 l.

In conventional ventilators, resistance is usually determined by this method. In addition, there are various other methods for determining resistance that are based on intermittent or superimposed measurements.

In order to be able to fully capture and describe the properties of a ventilated lung, it is desirable not only to precisely measure dynamic compliance, but also to accurately determine inspiratory and expiratory resistance. This is required in order to be able to ventilate especially critically ill patients (whose lungs are affected) in a personalized manner and as gently as possible in the region of optimal compliance, i.e., between a so-called lower inflection point, at which compliance maximally increases during inspiration in terms of optimal recruitment of the lung tissue, and a so-called upper inflection point, at which compliance maximally decreases during inspiration owing to increasing overstretching in the lung tissue.

However, resistance arises not only from the gas flow-dependent resistance of the airways (airway-related; e.g., cross-section of the airways, turbulence), but also from resistances in the tissue (tissue-related; e.g., due to mass inertia, shearing, friction, viscoelasticity).

Mass inertia effects play a role especially at the start of inspiration and expiration, when tissue (both the lung itself and the surrounding/adjacent tissue) has to be accelerated and decelerated owing to the increase or decrease in lung volume. Shearing can occur within (especially functionally inhomogeneous) lung tissue (so-called “shear stress”), whereas friction can occur at interfaces such as, for example, the sliding layer of the outer and inner pulmonary pleurae (pleura parietalis and pleura visceralis), which moreover increases in (surface) size during inspiration and decreases in (surface) size during expiration. Viscoelastic effects arise, inter alia, from a differing blood volume in the pulmonary vascular system during inspiration and expiration, making the lung differently resistive.

Within the lung, there are not only differently compliant (expandable) lung compartments, but there are also, with regard to resistance, lung compartments which have a lower or higher airway-related or tissue-related resistance. This inevitably means that an external observer only ever sees a global picture of compliance and resistance.

However, differentiation of resistance into an airway-related component and tissue-related component is of clinical interest: on the basis of the airway-related component, it is possible in principle to calculate the (global) alveolar pressure plot (in the alveoli). Moreover, an increased airway-related resistance (in contrast to the tissue-related component) is amenable to drug therapy. By contrast, tissue-related resistance points to changes in the (lung) tissue. Thus, tissue-related resistance can be used as a diagnostic, therapeutic or even prognostic parameter.

In the literature, the proportion of the tissue-related resistance in the (total) resistance measured at peak (inspiratory) pressure is estimated at about 25%, i.e., about 75% of the (total) resistance is attributable to the airway-related component. These data are usually based on invasive measurement methods (e.g., esophageal pressure measurements).

It is an object of the present invention to solve (at least in part) the problems cited with regard to the prior art. In particular, a ventilator and a method at least for measurement or determination of the tissue-related resistance in the airway of a patient, preferably for (differentiated) measurement or determination of the airway-related and tissue-related resistance and for ascertainment of the (global) alveolar pressure or pressure plot, by means of a ventilator, shall be proposed. In particular, a ventilator by means of which indices are definable and determinable shall be proposed.

A ventilator having the features as claimed in claim 1 and a method having the features as claimed in claim 12 contribute to achieving these objects. Advantageous developments are the subject matter of the dependent claims. The features individually stated in the claims are combinable with one another in a technologically feasible manner and can be supplemented by explanatory facts from the description and/or details from the figures, showing further embodiment variants of the invention.

There is proposed a ventilator, at least comprising a gas supply device and a gas discharge device, for supplying an (inspiratory) fluid flow to an airway of a patient and for discharging an (expiratory) fluid flow from the airway (of the patient) back into the ventilator or to an environment, a pressure sensor for measuring a pressure in the airway, and a control device for operating the ventilator. A fluid flow is adjustable to a constant value at least during an inspiration process and an expiration process. The control device is configured to operate the ventilator and to carry out a method, especially a measurement method, comprising at least the following steps:
a) carrying out an inspiration process with a constant first fluid flow by means of the gas supply device,
b) stopping the (inspiratory) first fluid flow by means of the gas supply device at a first time point, and at the same time
c) (measuring or) determining a first pressure difference between a first pressure present at the first time point of stopping and a second pressure occurring after a time interval by means of the pressure sensor; and
d) carrying out an expiration process with a constant second fluid flow by means of the gas discharge device,
e) stopping the (expiratory) second fluid flow by means of the gas discharge device at a second time point, and at the same time
f) (measuring or) determining a second pressure difference between a third pressure present at the second time point of stopping and a fourth pressure occurring after a time interval by means of the pressure sensor;
g) defining and providing (or forming) a difference between the first pressure difference and the second pressure difference as a first index which is usable or used for determination of at least a tissue-related resistance of the patient.

Preferably, the first fluid flow is constant over the entire inspiration process, but especially in the second half of the inspiration process. Preferably, the second fluid flow is constant over the entire expiration process, but especially in the second half of the expiration process. In particular, the (inspiratory) first fluid flow and the (expiratory) second fluid flow are equal in absolute value.

In particular, what is meant here by constant or equal in absolute value is that there can be deviations of up to 10%, preferably up to 5%, particularly preferably up to at most 1%, compared to the highest value of the fluid flow.

In particular, in the case of the ventilator proposed here, the patient is ventilated solely via the ventilator. In particular, the fluid flow is thus admitted to the airway of the patient solely via the ventilator (during inspiration and expiration). In particular, there is thus no fluid flow that is not initiated or generated by the ventilator. In particular, the ventilator comprises for this purpose a lumen for inspiration and a lumen for expiration. In particular, a common lumen (e.g., a ventilation catheter) is provided, so that the fluid flow is supplied to the airway or discharged from the airway only through one lumen.

In particular, the pressure sensor is arranged endotracheally (i.e., in the trachea). This makes it possible to ascertain the pressure inside the airway of the patient.

In particular, the pressure sensor is arranged at the distal end of a ventilation catheter, which is located in the airway of the patient as part of the ventilator.

The pressure sensor can optionally also be arranged at a distance from the patient, instead of endotracheally. The tracheal pressure should then be mathematically determinable. However, such an arrangement of the pressure sensor can give rise to inaccuracies which can impair the measurements described here.

In particular, the ventilation catheter has a dead space volume (i.e., the volume remaining in the ventilation catheter during an inspiration or expiration process) of at most 100 ml, in particular of at most 50 ml.

It has been observed that the (total) resistance increases when VT increases but gas inflow is the same, presumably due to an increase in the tissue-related resistance. This seems plausible for the following reason: the small pressure drop from the small bronchi or bronchioles (having smooth muscles), which largely determine the airway-related resistance, to the alveoli makes it possible to assume an airway-related resistance largely independent of the ventilation pressures, since higher pressures in the small bronchi or bronchioles (having smooth muscles) are also necessarily associated with higher pressures in the dependent alveoli surrounding the small bronchi or bronchioles (having smooth muscles).

In particular, however, the airway-related resistance seems to decrease slightly during the inspiration process. This can be explained by a cross-sectional enlargement (though small) of the bronchi with increasing pressure, which more than compensates for the resistance-increasing effect of an increasing length of the small bronchi or bronchioles (having smooth muscles) during inspiration. In this regard, reference is made to the Hagen-Poiseuille equation, which states that changes in radius affect gas flow (fluid flow) to the fourth power, but changes in length affect gas flow (fluid flow) only proportionally.

Even if the size of the pressure drop from the small bronchi or bronchioles (having smooth muscles) to the alveoli is necessarily gas flow-dependent, and a relatively greater expansion and hence cross-sectional enlargement of the small bronchi or bronchioles (having smooth muscles) can thus occur at higher gas flows, higher airway pressures thus lead at most to a decrease, but not an increase, in the airway-related resistance.

It can therefore be plausibly assumed that the increase in the inspiratory (total) resistance at higher tidal volumes but at the same gas inflow (fluid flow during inspiration) is largely based on an increase in the tissue-related resistance.

Conversely, one can expect that, in the case of an expiratory outflow of the respiratory gas (fluid flow during expiration) and a thereby decreasing lung volume, the proportion of the tissue-related resistance in the (total) resistance decreases again and eventually (virtually) disappears in the case of complete expiration or reaches a minimum in the case of PEEP.

In particular, what is thus assumed for step g), or is defined for the method by the control device, is that the tissue-related resistance is negligible in the end-expiratory state and is maximal in the end-inspiratory state.

In particular, by solely carrying out steps d) to f), and when the third pressure corresponds to an end-expiratory pressure, an airway-related resistance of the patient is determinable (since tissue-related resistance is negligible in the end-expiratory state). In particular, it is thus possible, in the case of end-expiratory pressure (as the third pressure), to carry out only steps d) to f) once, or preferably repeatedly during ventilation, in order to (directly) ascertain the airway-related resistance.

In particular, by carrying out steps d) to f), and when the third pressure corresponds to an end-expiratory pressure, a second index is defined and provided and an airway-related resistance of the patient is thus determinable.

In particular, what is additionally assumed for step g), or is defined for the method by the control device, is that the tissue-related resistance increases (approximately) linearly during the inspiration process between the end-expiratory state and the end-inspiratory state and decreases (approximately) linearly again during the expiration process between the end-inspiratory state and the end-expiratory state.

In particular, an airway-related resistance (see the 1st calculation example below) and (by conversion to the known constant fluid flow) the inspiratory and expiratory pressure drop are also determinable by means of the control device in step g).

Thus, it is assumed here in particular or defined for the method by the control device that the (total) resistance measured end-expiratorily is more or less only airway-related and thus lower than the (total) resistance which is measured end-inspiratorily after the peak pressure (PIP) has been reached and which also includes the tissue-related resistance. This in turn corresponds to the difference between (total) resistance measured during inspiration and during expiration.

In order to be able to ascertain the airway-related and/or tissue-related resistance or the first index and/or the second index, the following is particularly necessary or useful:
i. gas flow (fluid flow) during inspiration (inspiration process) and expiration (expiration process) that is relatively stable or relatively constant and is especially equal in absolute value. It should be noted that, in the case of varying fluid flow (e.g., with pressure- or volume-controlled ventilation), conversion of the measured resistance to the same gas flow is at least complex and laborious, especially in the case of (highly) dynamic flow profiles (with unequal gas flow);
ii. ideally tracheal (in the windpipe) measurement of pressure during inspiration; i.e., for example, of the pressure drop after stopping the inspiratory gas inflow (e.g., immediately after the set peak inspiratory pressure has been reached or the desired VT to be supplied has been reached) from the peak inspiratory pressure to the end-inspiratory (plateau) pressure;
iii. ideally tracheal (in the windpipe) measurement of pressure during expiration; i.e., for example, of the pressure rise after stopping the expiratory gas outflow (e.g., immediately after returning to the set end-expiratory pressure) from the end-expiratory pressure to the end-expiratory (plateau) pressure.

For the following 1st calculation example, the first pressure difference was measured or determined in the end-inspiratory state and the second pressure difference was measured or determined in the end-expiratory state. In the end-inspiratory state, the measured (total) resistance is thus composed of a maximum of the tissue-related resistance and the airway-related resistance (which is especially constant over V_{T}). In the end-expiratory state, the measured (total) resistance comprises especially only the airway-related resistance, since the tissue-related resistance can be disregarded.

1st calculation example:

An end-inspiratory (total) resistance (i.e., sum total of airway-related and tissue-related resistance) of 8 mbar/I/s, measured as the first pressure difference, corresponds, at a gas flow of 12 I/min (i.e., 0.2 I/s), to a (total) resistance of 1.6 mbar/0.2 I/s. If a (total) resistance of 4 mbar/I/s at the same flow of 12 I/min (i.e., 0.8 mbar/0.2 I/s) is measured end-expiratorily as the second pressure difference, assuming that “end-expiratory/end-expiratorily” means that the resistance is then only airway-related and the tissue-related resistance is negligible, the airway-related resistance is thus 0.8 mbar/0.2 I/s (corresponding to the end-expiratory measurement) and the (maximal) tissue-related resistance is likewise 0.8 mbar/0.2 I/s, i.e., 4 mbar/I/s (corresponding to the end-inspiratorily measured (total) resistance minus the airway-related resistance).

A value of 1.6 mbar is measured here by means of the pressure sensor as the first pressure difference between the end-inspiratory first pressure and the second pressure occurring after a time interval, at a fluid flow of 12 I/min (i.e., 0.2 I/s) that is constant and known and is present up to measurement of the first pressure difference.

On basis of the known fluid flow, this determined or measured first pressure difference can be converted to the end-inspiratory (total) resistance, i.e., to the airway-related and tissue-related resistance, in this case to a value of 8 mbar/I/s. The value of 8 mbar can be used as a value of the first pressure difference that is normalized to a fluid flow of 1 I/s.

Furthermore, a value of 0.8 mbar is measured by means of the pressure sensor as the second pressure difference (second index) between the end-expiratory third pressure and the fourth pressure occurring after a time interval, at a fluid flow of 12 I/min that is constant and the same and is present up to measurement of the second pressure difference.

On basis of the known fluid flow, this determined or measured second pressure difference can be converted to the end-expiratory (total) resistance, i.e., to the airway-related resistance, thus in this case to a value of 4 mbar/I/s. The value of 4 mbar can be used as a value of the second pressure difference that is normalized to a fluid flow of 1 I/s.

In accordance with step g), the (maximal) tissue-related resistance is determined from the difference between the first pressure difference and the second pressure difference, in this case: 1.6 mbar−0.8 mbar=0.8 mbar (or, normalized, 8 mbar−4 mbar=4 mbar). This difference, i.e., the value of 0.8 mbar, is defined and provided as the first index by the control device. Taking into account the fluid flows that are constant and the same during inspiration and expiration, this means that the (maximal) tissue-related resistance is 0.8 mbar/0.2 I/s, i.e., a normalized 4 mbar/I/s (corresponding to the end-inspiratorily measured (total) resistance minus the airway-related resistance).

For step g) and the definition or provision of the difference between the pressure differences, it is especially necessary to convert the pressure differences to normalized pressure differences, i.e., the pressure differences should either (preferably) be determined or measured at the same fluid flows or be converted thereto (see the 1st calculation example).

When assuming or defining an airway-related resistance that remains largely the same during inspiration and expiration and a tissue-related resistance that gradually increases (approximately) linearly during inspiration and gradually decreases (approximately) linearly again during expiration (which appears permissible in the case of ventilation in the region of optimal or maximal compliance and under the prerequisite of slow and even changes in pressure and volume over time), it is possible to derive the (global) alveolar pressure or pressure plot.

To this end, what must be first ascertained or what the control device must first ascertain, by converting the airway-related resistance (i.e., the (total) resistance minus the proportion due to tissue-related resistance) to the respective (current) gas flow, is the pressure drop that is responsible for the gas flow from trachea to alveoli (i.e., from the windpipe in the direction of the alveoli) during inspiration or for the gas flow from alveoli to trachea (i.e., from the alveoli in the direction of the windpipe) during expiration. In particular, the pressure difference (pressure drop) between the tracheal pressure and the alveolar pressure thus arises from the airway-related resistance and the constant fluid flow, i.e., from the product of airway-related resistance (as a multiplier) and the fluid flow (as a multiplicand) (in the 1st calculation example, this is 0.8 mbar, namely 0.8 mbar/0.2 I/s·0.2 I/s or 4 mbar/I/s·12 l/min).

This pressure drop is then subtracted from the pressure measured in the trachea during inspiration or added to the pressure measured in the trachea during expiration, and the (global) alveolar pressure (at a given time point) or pressure plot (over time) is thus ascertained.

In particular, a regression analysis, especially a linear regression analysis, is performable by means of the control device at least to determine the tissue-related resistance. Use is preferably made of a linear function to describe the change in tissue-related resistance between the end-inspiratory state and the end-expiratory state. The control device is suitably designed especially to carry out the regression analysis.

2nd calculation example:

On the basis of a (tracheal) pressure measurement, an end-inspiratorily measured first pressure difference and a thus determined (total) resistance of 6 mbar/I/s at a gas flow of 15 I/min (i.e., 0.25 I/s) results in a (total) resistance of 1.5 mbar/0.25 I/s. An end-expiratorily measured second pressure difference and thus determined (total) resistance (corresponding to the airway-related resistance) of 4 mbar/I/s corresponds, at the same gas flow, to 1 mbar/0.25 I/ s. The maximal tissue-related resistance thus corresponds to 0.5 mbar/0.25 I/s. At half V_{T} (and with linear regression of half of the tissue-related resistance), a pressure difference of 1.25 mbar in each case is calculated for inspiration and expiration, namely from an airway-related resistance of 1 mbar/0.25 I/s and a tissue-related resistance of 0.25 mbar/0.25 I/s.

As explained in connection with the 1st calculation example, a value of 1.5 mbar is measured as the first pressure difference by means of the pressure sensor. A value of 1 mbar is measured as the second pressure difference. From the second pressure difference as the second index, it is possible to directly derive the normalized airway-related resistance of 4 mbar/I/s. The maximal tissue-related resistance of 0.5 mbar/0.25/s or a normalized 2 mbar/I/s can thus be determined from the difference between the pressure differences.

In particular, it is possible to ascertain, from a measurement or determination of pressure differences during the inspiration process (and not only when the peak inspiratory pressure has been reached) and/or the expiration process (and not only when the PEEP has been reached), at least the tissue-related resistance over the respective pressure interval. In particular, the position of the time points in relation to the end-inspiratory state and end-expiratory state is taken into account (e.g., by means of the control device) and the proportion of the tissue-related resistance present at the respective time point is determined, for example on the basis of a regression analysis.

In particular, at least the second pressure or the fourth pressure is mathematically determinable (by the control device). It can be assumed that the plot of the pressure drop occurring during inspiratory measurement and pressure rise occurring during expiratory measurement typically runs asymptotically in each case. It is thus possible, from an initial plot of the pressure drop or pressure rise, to mathematically determine or at least estimate the end value of the pressure that occurs at a later time point. Optionally, it is also possible to actually wait for this pressure drop or pressure rise for a patient and to thus mathematically approximate the plot, so that, as the ventilation of the patient proceeds, the end value of the pressure that occurs can be determined on the basis of the approximately ascertained plot.

Various ventilation methods are known: in the case of (classic) volume-controlled ventilation (VCV), the V_{T} and the ventilation rate (and optionally the PEEP) are the primary setting parameters. During the insufflation phase, the V_{T} is typically supplied with a constant gas flow (fluid flow). The gas inflow is generally not directly adjustable and results from the duration of the insufflation phase and the duration of the subsequent inspiratory plateau pressure phase in which no gas is supplied. In the case of (classic) volume-controlled ventilation, the airway pressures depend on the set V_{T} and the pulmonary conditions of the patient.

In the case of pressure-controlled ventilation (PCV), an initially high inspiratory gas flow (fluid flow) is continuously reduced, while the pressure rise is measured by the ventilator. The inspiratory pressure (and optionally the PEEP) is thus the target parameter and control variable. The V_{T} results from the pressures set on the ventilator and the pulmonary conditions of the patient. Direct adjustment of the inspiratory gas flow is also not possible; however, it is measured.

VCV and PCV (and all ventilation methods derived therefrom) rely on uncontrolled expiration. Thus, the expiratory gas outflow (fluid flow) depends on the restoring forces of the thorax/lung system and the expiratory (total) resistance, changes constantly during expiration (gradually decreases) and approaches zero toward the end of expiration. The construction of a (global) alveolar pressure-time curve therefore requires not only an accurate, continuous measurement of expiratory gas flow, but also complex mathematics that can be used to estimate the respective pressure drop from alveoli to trachea at different times during expiration (e.g., “Verfahren zur atemzugweisen kontinuierlichen Bestimmung der intratidalen dynamischen Atemmechanik mittels gleitender multipler Regressionsanalyse” [“Method for breath-by-breath continuous determination of intratidal dynamic respiratory mechanics by means of sliding multiple regression analysis”] according to DE 10 2006 025 809.6). However, a simple calculation based on the mean expiratory gas outflow is very inaccurate and therefore unsuitable.

Flow-controlled ventilation (FCV; e.g., DE 10 2016 103 678.1 and DE 10 2016 109 528.1) is a mode of ventilation that has now also been clinically implemented, in which (in contrast to conventional ventilators) the gas flow is controlled and regulated not only during inspiration, but also during expiration. In the case of FCV, the expiratory gas outflow corresponds especially to the inspiratory gas inflow; this results in a ratio of inspiration to expiration of preferably 1:1. The gas flow (fluid flow) is stable or constant (i.e., does not exhibit any relevant change in absolute value) and is preferably just high enough for normoventilation to be achieved in the patient. Especially at the start of expiration, i.e., starting from the peak inspiratory pressure, the second fluid flow is preferably reduced (e.g., by a resistance). During expiration and especially toward the end of expiration, i.e., toward the end-expiratory pressure, the second fluid flow is then increasingly assisted (e.g., by suction).

Only one other ventilation method in which the expiratory gas outflow can be modulated is known, this method being experimental (but not yet clinically available): in the case of “FLow-controlled EXpiration” (FLEX; see Minerva Anestesiologica 80 (1): 19-28 (2014)), some control of expiration is achieved by a passive, dynamic resistor, which is arranged in the exhalation limb of a conventional ventilator and the resistance of which is gradually reduced during expiration. Depending on the restoring forces of the thorax/lung system and the expiratory (total) resistance, this system can modulate the gas outflow, but it cannot create and maintain a (largely) stable expiratory gas outflow. An I:E ratio of 1:1 and thus an inspiratory and expiratory gas flow (fluid flow) that is equal in absolute value is not possible either.

Compared to FLEX, the advantage of FCV is that the expiratory fluid flow is actively regulated (in the sense of a stable or constant gas flow) and thus known.

This can, for example, be achieved with an active, dynamic resistor (e.g., combination of a resistance element with suction, e.g., by a gas-flow reversal element, e.g., known from DE 10 2007 013 385.7). For example, in a (temporally) first half of expiration, the particularly high fluid flow due to the restoring forces of the thorax(lung system is initially reduced by a resistance element. In a (temporally) second half of expiration, when the restoring forces become smaller and the fluid flow would thus usually gradually decrease, the fluid flow is increased by suction (e.g., a negative-pressure connection or the like) and kept constant overall.

Especially in the (temporally) second half of expiration, the gas outflow (second fluid flow) is thus very stable and can be adjusted in terms of absolute value especially according to the inspiratory gas inflow (first fluid flow), i.e., especially over the entire expiration process.

Compared to ventilation methods that operate with gas flow which decelerates during inspiration and/or expiration (e.g., VCV, i.e., volume-controlled ventilation, with gas flow regulated only during inspiration, or PCV, i.e., pressure-controlled ventilation, with gas flow likewise regulated only during inspiration), FCV creates optimal conditions for the measurements and calculations described here.

For a very simple and accurate calculation of the (global) alveolar pressure or pressure plot, preference is given to an inspiratory and expiratory fluid flow which is stable or constant in each case and especially equal in absolute value, not least because of unavoidable latency times in the otherwise necessary measurement of gas flow, signal processing and subsequent regulation and control of gas flow. Only FCV meets these requirements.

In particular, the second half of inspiration and the second half of expiration is particularly advantageous, since fluid flow conditions are (very) stable here in FCV and the effects (e.g., mass inertia effects) relevant in the first halves (especially at the start of inspiration and expiration or when changing from inspiration to expiration and from expiration to inspiration) are hardly significant. A good representation of the (global) alveolar pressure or pressure plot can therefore be obtained from the conversion of the (preferably tracheally) measured pressure, taking into account the ascertained airway-related resistance.

The (global) alveolar pressure or pressure plot can also be estimated approximately on the basis of the ascertained (total) resistance. However, the specific gas flow conditions of FCV (gas flow during inspiration and expiration that is continuous and constant and especially equal in absolute value) are again a prerequisite for a good approximation.

3rd calculation example:

In view of the typically low gas flow in FCV of not more than 15 I/min (i.e., 0.25 I /s), there is an error of not more than 1 mbar for the (global) alveolar pressure or pressure plot even at an increased (total) resistance of a normalized 8 mbar/I/s (i.e., 2 mbar/0.25 I/s) by nonconsideration of the tissue-related resistance (assuming a proportion of the tissue-related resistance in the (total) resistance that is increased to 50%).

For other reasons as well (e.g., mechanical- and energy-related reasons), it is appropriate to ventilate with slow and even changes in pressure and volume using the region of individually optimal, i.e., maximal, compliance.

FCV is especially intended for controlled ventilation with maximal lung protection, but not for assisting spontaneous breathing, since this requires distinctly higher gas flows.

Differences with respect to gas distribution in the lung can be minimized (within what is physically possible) especially through a fluid flow which is as low and stable as possible and is equal in absolute value during inspiration and expiration. CT scans and electrical impedance tomography have already demonstrated an overall better and more homogeneous ventilation of both healthy and diseased lungs by FCV.

It is thus an aim of the invention to describe a ventilator or method that allows a simple and accurate (within what is physically possible) determination of indices or the airway-related and tissue-related resistance and, on the basis thereof, the ascertainment of the (global) alveolar pressure or pressure plot during the controlled ventilation of a patient.

The ventilator is, for example, a ventilator which ventilates with a fluid flow which is continuous (without any relevant pauses) and is stable or constant and is equal in absolute value during inspiration and expiration (and thus an I:E ratio of typically 1:1), preferably in the region of optimal or maximal compliance. The fluid flow is just high enough for normoventilation or the desired degree of carbon dioxide elimination or exhalation to be achieved in the patient.

A user or preferably an (automatically operating) control device of the ventilator can, taking into account an ascertained or additionally estimated plot of at least a section of a compliance curve in a pressure-volume diagram, determine a position of a pressure interval having the pressures Pₗ and P_{E} and set these pressures on the ventilator (e.g., PIP as Pₗ or as the first pressure and PEEP as P_{E} or as the third pressure), so that at least a ventilation process, i.e., an inspiration and/or an expiration, occurs between these pressures Pₗ and P_{E} and an absolute value of the compliance of this ventilation process is as large as possible as a result over the resultant V_{T}. Alternatively, the position of a pressure interval having the pressures Pₗ and P_{E} can also be ascertained in a volume-pressure diagram. Moreover, the ventilation process should be set in such a way that a minute volume required for normoventilation can be supplied and discharged at maximum possible compliance, since an (optimally) large V_{T} with an (optimally) low ventilation rate increases the efficiency of carbon dioxide elimination and the airway or tissue of the patient is thus stressed as little as possible.

Although the inspiratory and expiratory fluid flow can differ in absolute value, what is especially envisaged is just a deviation of the current fluid flow from a set fluid flow or average fluid flow by at most 10%, preferably at most 5%, particularly preferably at most 1%, during the inspiration process and during the expiration process. In particular, a relevant deviation is also possible between the inspiration process and the expiration process. In particular, however, the ratio between inspiration process and expiration process is 1:1, i.e., the fluid flow is constant and is equal in absolute value for the inspiration process and the expiration process.

The ventilator is technically designed especially for a continuous measurement of pressure, preferably in the trachea, and is programmed or programmable in such a way that, either by the user or in fixed cycles during a sufficiently long pause of fluid flow, what is or can be done during the inspiration process (first time point), especially after the peak inspiratory pressure (PIP) has been reached, is measurement or determination of the pressure drop occurring in the subsequent time interval and what is or can be done during the expiration process (second time point), especially after the end-expiratory pressure (PEEP) has been reached, is measurement or determination of the pressure rise occurring in the subsequent time interval.

The inspiratory and expiratory measurements can fall into the same ventilation cycle (i.e., an inspiration process and a directly following expiration process), be distributed over two consecutive ventilation cycles or preferably be separated by a few (normal) ventilation cycles.

By means of the ventilator or by means of the method for differentiated measurement of the airway-related and tissue-related resistance and for ascertainment of the (global) alveolar pressure or pressure plot, it is possible to ascertain and optionally output (e.g., on a display of the ventilator) the airway-related and tissue-related resistance on the basis of these pressure measurements and of the respective (known) inspiratory and expiratory fluid flow.

From the airway-related resistance and the respective (known) inspiratory and expiratory fluid flow, it is then possible to calculate the inspiratory and expiratory pressure drop (even during ventilation) and to optionally output it (e.g., on the display of the ventilator). Lastly, the (global) alveolar pressure or pressure plot can also be calculated and optionally output (e.g., on the display of the ventilator).

By means of the ventilator and/or the described method, it is possible to determine and differentiate the airway-related and tissue-related resistance even over sections of (preferably optimal, i.e., maximal) compliance especially by means of temporary stopping of the fluid flow during inspiration and expiration.

The combination of a measurement after the set peak inspiratory pressure has been reached at the first time point with an interim measurement before the set end-expiratory pressure has been reached at the second time point makes it possible in particular to determine and differentiate the airway-related and tissue-related resistance even more precisely and to accordingly also calculate the (global) alveolar pressure or pressure plot even more precisely.

The same is also achieved by the combination of an interim measurement at the first time point before the set peak inspiratory pressure has been reached with a measurement at the second time point after the set end-expiratory pressure has been reached.

Especially in the case of interim measurements, i.e., during inspiration and before the peak inspiratory pressure has been reached or during expiration and before the end-expiratory pressure has been reached, it should be borne in mind that, in the case of FCV, gas flow conditions are (very) stable or constant especially in the second half of inspiration and in the second half of expiration and measurement conditions are therefore then particularly advantageous.

Also possible is a measurement with an intermittently (i.e., only for the relevant cycle) lower or higher peak pressure and/or an intermittently (i.e., only for the relevant cycle) lower or higher end-expiratory pressure. However, this should preferably be done in the region of maximal compliance, otherwise (regional) overexpansion can occur at an excessively high peak pressure or (regional) derecruitment of lung tissue can occur at an excessively low end-expiratory pressure.

Preferably, the (global) alveolar pressure or pressure plot is calculated on the basis of the temporal second half of inspiration and the temporal second half of expiration, since the gas inflow (inspiratory fluid flow from the ventilator to the airway) and gas outflow (expiratory fluid flow from the airway back into the ventilator or into the environment) are then both very stable (in terms of absolute value) and mass inertia effects, which occur especially in inhomogeneous lungs at the start of inspiration and expiration or when changing from inspiration to expiration and from expiration to inspiration, and resultant shear effects are hardly relevant.

In particular, at least in step b), the (inspiratory) first fluid flow is stopped when a peak inspiratory pressure has been reached and/ or, in step e), the (expiratory) second fluid flow is stopped when an end-expiratory pressure has been reached (see, for example, also the 1st calculation example).

In particular, the first pressure difference (first pressure corresponds to peak inspiratory pressure) gives rise to a (total) resistance as the sum total of an airway-related resistance and a maximum of a tissue-related resistance, and the second pressure difference (third pressure corresponds to end-expiratory pressure) gives rise to the (total) resistance composed (only) of the airway-related resistance (because the tissue-related resistance is negligible or is not taken into account).

In particular, the pressure difference (pressure drop) between the tracheal pressure and the alveolar pressure arises from the airway-related resistance and the constant fluid flow, i.e., from the product of airway-related resistance and the fluid flow. From this, it is possible in particular to calculate the alveolar pressure or (over time) the alveolar pressure plot.

Alternatively, it is also possible on the basis of the inspiratory (total) resistance (i.e., without taking into account the tissue-related resistance) to estimate quite well the (global) alveolar pressure or pressure plot (see, for example, also the 3rd calculation example).

There is further proposed a method for determining at least a tissue-related resistance of a patient by means of a ventilator, especially by means of the described ventilator.

The ventilator at least comprises a gas supply device and a gas discharge device, for supplying an (inspiratory) fluid flow to an airway of a patient and for discharging an (expiratory) fluid flow from the airway (of the patient) back into the ventilator or to an environment, a pressure sensor for measuring a pressure in the airway, and a control device for operating the ventilator. A fluid flow is adjustable to a constant value at least during an inspiration process and an expiration process. The control device is suitably designed or configured to carry out a method (or a measurement method) comprising at least the following steps:
a) carrying out an inspiration process with a constant first fluid flow by means of the gas supply device,
b) stopping the (inspiratory) first fluid flow by means of the gas supply device at a first time point, and at the same time
c) (measuring or) determining a first pressure difference between a first pressure present at the first time point of stopping and a second pressure occurring after a time interval by means of the pressure sensor; and
d) carrying out an expiration process with a constant second fluid flow by means of the gas discharge device,
e) stopping the (expiratory) second fluid flow by means of the gas discharge device at a second time point, and at the same time
f) (measuring or) determining a second pressure difference between a third pressure present at the second time point of stopping and a fourth pressure occurring after a time interval by means of the pressure sensor;
g) defining and providing (or forming) a difference between the first pressure difference and the second pressure difference as a first index and determining at least a tissue-related resistance of the patient.

The above (nonexhaustive) division of the method steps into a) tog) is primarily intended for the purpose of differentiation only and does not impose any order and/or dependency. The frequency of the method steps, for example during the configuration and/or operation of the ventilator, can vary, too. It is also possible that method steps temporally overlap at least in part. Very particularly preferably, method steps a) to c) and method steps d) to f) take place alternately one after the other. However, it is also possible to repeat method steps a) to c) or method steps d) to f) multiple times in each case. Step g) can in particular be carried out after steps a) to f) have been carried out once or else after steps a) to f) or at least steps a) to c) or d) to f) have been carried out multiple times. Step g) can be conditional and may only be carried out if steps a) to f) have been carried out at least once.

In particular, steps a) to g) are carried out in the order cited.

In particular, at least steps a) to c) during an inspiration process or steps d) to f) during an expiration process are in each case carried out multiple times together with step g).

In particular, steps a) to c) during an inspiration process and steps d) to f) during an expiration process are in each case carried out multiple times together with step g).

As described above, it is possible that steps b) and e) are carried out only once in each case, in the context of the method, for determination of the tissue-related resistance. For example, the first pressure can correspond to the peak inspiratory pressure (PIP) and the third pressure can correspond to the end-expiratory pressure (PEEP), though other pressure values can also be used for the first pressure and the third pressure.

In particular, it is possible, in the case of end-expiratory pressure (as the third pressure), to carry out only steps d) to f) once, or preferably repeatedly during ventilation, in order to define and provide a second index and to thus (directly) ascertain or determine the airway-related resistance.

It is also possible to stop the respective fluid flow multiple times and to determine the respective pressure difference at different pressure values. The respective fluid flow can be stopped in an inspiration process or expiration process of the same ventilation cycle or in different ventilation cycles.

By carrying out the stated steps multiple times, it is possible to determine more accurately the values sought for the tissue-related resistance and/or airway-related resistance, the pressure drop (from trachea to alveoli during inspiration or from alveoli to trachea during expiration), and the alveolar pressure or pressure plot.

In particular, it is assumed for step g) that the tissue-related resistance is negligible in the end-expiratory state and maximal in the end-inspiratory state and, in between, increases (approximately) linearly during the inspiration process and decreases (approximately) linearly during the expiration process.

There is further proposed a control device for a ventilator, especially for the described ventilator, that is (suitably) equipped, configured or programmed to carry out the described method.

The discussions relating to the ventilator are applicable especially to the method and to the control device and vice versa in each case.

Furthermore, the described method can also be carried out manually (in part) by a user or semiautomatically or (fully) automatically by a (separate) computer or with a processor of a control device.

Accordingly, there is also proposed a data processing system comprising a processor which is adapted, programmed and configured in such a way that it carries out the described method or some of the steps of the method (with or without communication with a user).

There can be provided a computer-readable storage medium comprising commands/algorithms which, when executed by a computer/processor, cause said computer/processor to carry out the described method or at least some of the steps of the method (with or without communication with a user).

The use of indefinite articles (“a”, “an”), especially in the claims and in the description reproducing said claims, should be understood as such and not as a numeral. Terms or components correspondingly introduced thereby are therefore to be understood in such a way that they are present at least once and can in particular, however, also be present multiple times.

As a precautionary measure, it should be noted that the numerals used here (“first”, “second”, . . . ) are primarily used (only) to distinguish between multiple objects, variables or processes of the same kind, i.e., in particular do not absolutely specify any dependency and/or order of these objects, variables or processes in relation to one another. Should a dependency and/or order be necessary, this is explicitly indicated here or it is obvious to a person skilled in the art upon studying the embodiment specifically described. If a component can occur multiple times (“at least one”), the description in relation to one of these components can apply equally to all or part of the plurality of these components; however, this is not mandatory.

The invention and the technical environment will be more particularly elucidated below with reference to the accompanying figures. It should be noted that the invention is not to be limited by the exemplary embodiments cited. In particular, unless explicitly stated otherwise, it is also possible to extract partial aspects of the facts elucidated in the figures and to combine them with other parts and findings from the present description. In particular, it should be noted that the figures and in particular the proportions depicted are only schematic. In the figures:

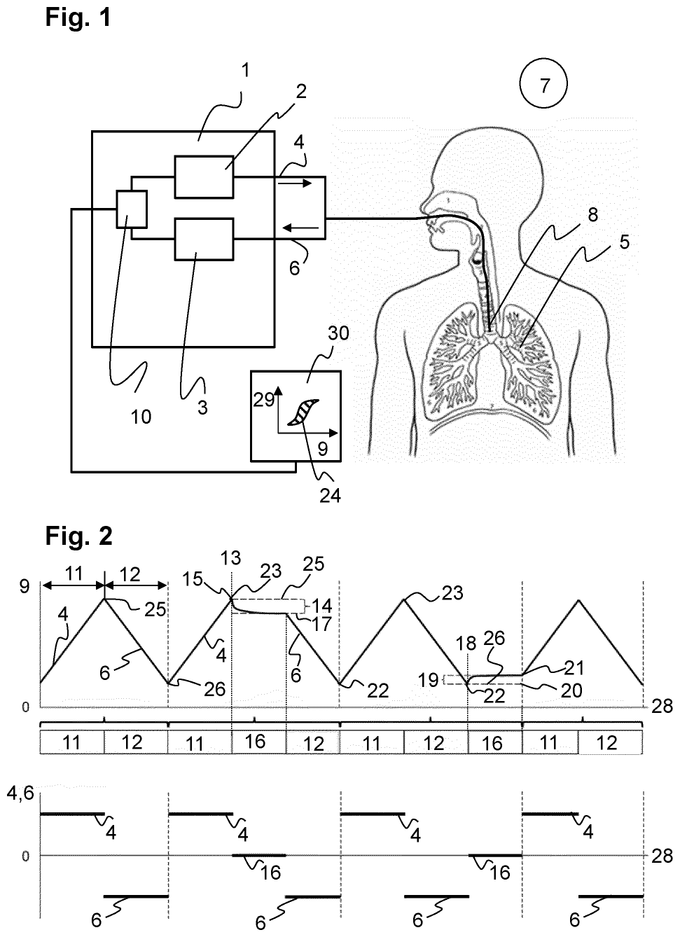

FIG. 1: shows a ventilator in operation;

FIG. 2: shows a first variant of the method;

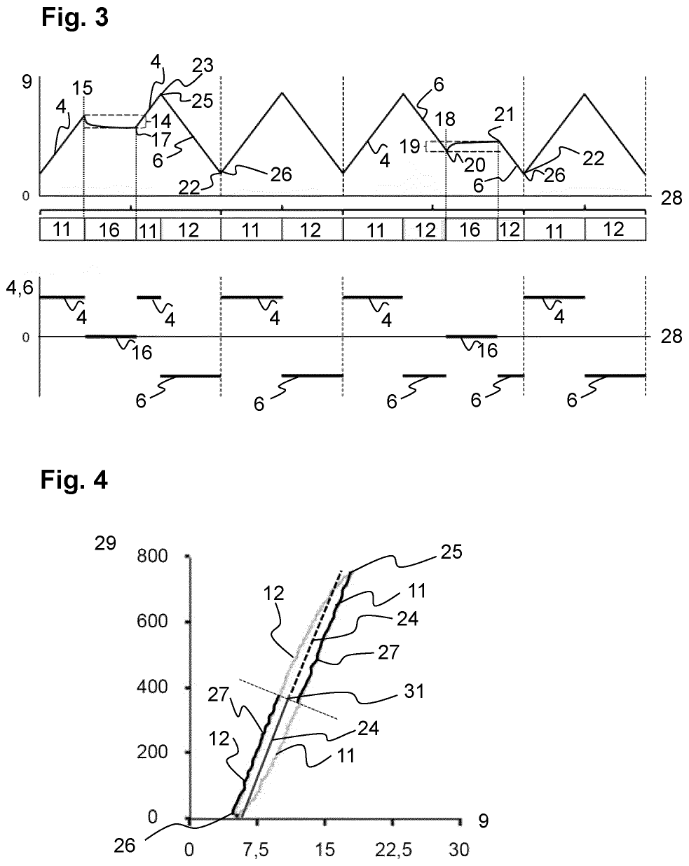

FIG. 3: shows a second variant of the method; and

FIG. 4: shows a pressure-volume diagram.

FIG. 1 shows a ventilator 1 in operation. The ventilator 1 comprises a gas supply device 2 and a gas discharge device 3, for supplying an (inspiratory) first fluid flow 4 to an airway 5 of a patient and for discharging an (expiratory) second fluid flow 6 from the airway 5 back into the ventilator 1 or into the environment 7, a pressure sensor 8 for measuring a pressure 9 in the airway 5, and a control device 10 for operating the ventilator 1. The fluid flow 4, 6 is adjustable to a constant value during an inspiration process 11 and an expiration process 12. The control device 10 is suitably designed to operate the ventilator 1 and to carry out the measurement method.

The pressure sensor 8 is arranged endotracheally. The pressure sensor 8 is located at the distal end of a ventilation catheter, which is arranged in the airway 5 of the patient as part of the ventilator 1.

The ventilator 1 also comprises a visualization device 30 (e.g., a display) on which the (total) resistance, airway-related resistance and tissue-related resistance, but especially also the current alveolar pressure 9 over time 28 and/or the plot 24 of alveolar pressure 9 and volume 29 (as a pressure-volume curve) are depictable.

FIG. 2 shows a first variant of the method. Multiple ventilation cycles are depicted in FIG. 2. The upper part of FIG. 2 depicts a graph in which pressure 9 is plotted on the vertical axis. Time 28 is plotted on the horizontal axis. The lower part of FIG. 2 depicts a graph in which fluid flow 4, 6 is plotted on the vertical axis. Time 28 is plotted on the horizontal axis.

The first ventilation cycle (far left) depicts a normal FCV cycle having a stable fluid flow 4, 6 that is equal (in absolute value) during inspiration and expiration and thus having a 1:1 ratio of inspiration process 11 to expiration process 12. The second ventilation cycle (second from the left) and the third ventilation cycle (second from the right) are measurement cycles: in the case of the second ventilation cycle, the first fluid flow 4 stops when the set peak inspiratory pressure 25 (first pressure 15) is reached. The pressure 9 then falls in the time interval 16 to an end-inspiratory (plateau) pressure, the second pressure 17. The thus ascertainable first pressure difference 14 in relation to the inspiratory first fluid flow 4 gives the (total) resistance (i.e., the sum total of airway-related and tissue-related resistance). In the case of the third ventilation cycle, the expiratory second fluid flow 6 stops when the set end-expiratory pressure 26 (third pressure 20) is reached. The pressure 9 then rises to a slightly higher end-expiratory (plateau) pressure, the fourth pressure 21. The thus ascertainable second pressure difference 19 in relation to the expiratory second fluid flow 6 gives the airway-related resistance. The tissue-related resistance is the difference between the pressure differences 14, 19 in relation to the inspiratory and expiratory fluid flow 4, 6, respectively. The fourth ventilation cycle (far right) is again a normal FCV cycle having a slightly shortened inspiration process 11 owing to the preceding measurement and the thus slightly increased starting pressure.

In accordance with step a) of the method or measurement method, an inspiration process 11 with a constant first fluid flow 4 is carried out in the second ventilation cycle. In accordance with step b), the first fluid flow 4 is stopped at a first time point 13, and at the same time, in accordance with step c), a first pressure difference 14 is measured or determined between a first pressure 15 present at the first time point 13 of stopping and a second pressure 17 occurring after a time interval 16. An expiration process 12 is then carried out. In the subsequent third ventilation cycle, an inspiration process 11 is first carried out, followed by carrying out, in accordance with step d), an expiration process 12 with a constant second fluid flow 6. In accordance with step e), the second fluid flow 6 is stopped at a second time point 18, and at the same time, in accordance with step f), a second pressure difference 19 is measured or determined between a third pressure 20 present at the second time point 18 of stopping and a fourth pressure 21 occurring after a time interval 16. In accordance with step g), the difference between the first pressure difference 14 and the second pressure difference 19 is formed and at least a tissue-related resistance of the patient is determined.

FIG. 2 corresponds to the 1st calculation example, in which the first pressure difference 14 is measured in the end-inspiratory state 23 and the second pressure difference 19 is measured in the end-expiratory state 22. In the end-inspiratory state 23, the measured (total) resistance is thus composed of a maximum of the tissue-related resistance and of the (especially constant) airway-related resistance. In the end-expiratory state 22, the measured (total) resistance comprises especially only the airway-related resistance, since the tissue-related resistance is disregarded.

FIG. 3 shows a second variant of the method. Reference is made to the discussions relating to FIG. 2.

Multiple ventilation cycles are depicted in FIG. 3. The upper part of FIG. 3 depicts a graph in which pressure 9 is plotted on the vertical axis. Time 28 is plotted on the horizontal axis. The lower part of FIG. 3 depicts a graph in which fluid flow 4, 6 is plotted on the vertical axis. Time 28 is plotted on the horizontal axis.

The second ventilation cycle (second from the left) and the fourth ventilation cycle (far right) are normal FCV cycles having a stable fluid flow 4, 6 that is equal (in absolute value) during inspiration and expiration and thus having a 1:1 ratio of inspiration process 11 to expiration process 12. The first ventilation cycle (far left) and fourth ventilation cycle are measurement cycles: in the case of the first ventilation cycle, the (inspiratory) first fluid flow 4 stops temporarily at a first pressure 15 before the set peak inspiratory pressure 25 is reached. The pressure 9 then falls to an (intermediate) inspiratory (plateau) pressure, the second pressure 17. The first pressure difference 14 in relation to the inspiratory first fluid flow 4 gives the (total) resistance (i.e., the sum total of airway-related and tissue-related resistance) at this first time point 13. In the case of the third ventilation cycle (second from the right), the (expiratory) second fluid flow 6 stops temporarily at a third pressure 20 before the set end-expiratory pressure 26 is reached. The pressure 9 then rises to a slightly higher (intermediate) expiratory (plateau) pressure, the fourth pressure 21. The second pressure difference 19 in relation to the expiratory second fluid flow 6 gives the airway-related (total) resistance (i.e., the sum total of airway-related and tissue-related resistance) at this second time point 18. The tissue-related resistance via the pressure difference 14, 19 when the fluid flow 4, 6 is temporarily stopped during inspiration and expiration is the difference between the first pressure difference 14 and the second pressure difference 19 in relation to the inspiratory and expiratory fluid flow 4, 6, respectively.

Assuming an airway-related resistance that remains largely the same during the inspiration process 11 and expiration process 12 and a tissue-related resistance that gradually increases (approximately) linearly during the inspiration process 11 and gradually decreases (approximately) linearly again during the expiration process 12 (which appears permissible in the case of ventilation in the region of optimal or maximal compliance and under the prerequisite of slow and even changes in pressure 9 and volume 29 over time 28), it is possible to derive the (global) alveolar pressure 9 or plot 24 of alveolar pressure 9.

To this end, what must be ascertained, by converting the airway-related resistance to the respective (current) fluid flow 4, 6, is the pressure drop that is responsible for the gas flow from trachea to alveoli (i.e., from the windpipe in the direction of the alveoli) during inspiration or for the gas flow from alveoli to trachea (i.e., from the alveoli in the direction of the windpipe) during expiration.

A regression analysis, especially a linear regression analysis, is performable by means of the control device 10 at least to determine the tissue-related resistance. Use is made of a linear function to describe the change in tissue-related resistance between the end-inspiratory state 23 and the end-expiratory state 22.

The control device 10 is suitably designed especially to carry out the regression analysis.

At least the tissue-related resistance can thus be ascertained from a measurement of the pressure differences 14, 19 during the inspiration process 11 (and not only when the peak inspiratory pressure 25 has been reached) and/or the expiration process 12 (and not only when the end-expiratory pressure 26 has been reached). In particular, the position of the time points 13, 18 in relation to the position of the end-inspiratory state 23 and the end-expiratory state 22 is taken into account and the proportion of the tissue-related resistance present at this time point 13, 18 is determined, for example on the basis of a regression analysis. In this connection, reference is made to the 2nd calculation example mentioned above.

FIG. 4 shows a pressure-volume diagram. Volume 29 (in ml) is plotted on the vertical axis. Pressure 9 (in mbar) is plotted on the horizontal axis. Reference is made to the discussions relating to FIGS. 1 to 3.

FIG. 4 is based on a pressure-volume curve of an optimally ventilated patient. The (global) alveolar pressure 9 or the plot 24 of the alveolar pressure 9 can be estimated quite well just on the basis of the inspiratory (total) resistance. To this end, use is made of the second half 27 of the inspiratory pressure-volume curve and the second half 27 of the expiratory pressure-volume curve, since fluid flow conditions are (very) stable here in FCV and the effects (e.g., mass inertia effects and resultant shear effects) relevant in the first halves (especially at the start of the inspiration process 11 and expiration process 12 or when changing from inspiration process 11 to expiration process 12 and inspiration process 11 again) are hardly significant.

In this example, the inspiratory and expiratory fluid flow 4, 6 is 11 I/min (=0.183 I/s) in each case. The inspiratory (total) resistance is 5.7 mbar/I/s or 1.05 mbar/0.183 I/s (based on the fluid flow 4, 6). The width of the pressure-volume curve in the middle (along the dashed line intersecting the curve) is 2.1 mbar. The (maximal) inspiratory pressure drop from trachea to alveoli estimated at 1.05 mbar or the plot 24 thereof is drawn in as a dashed parallel line in relation to the straight second half 27 of the inspiratory pressure-volume curve (traced here in black), i.e., of the inspiration process 11. The expiratory pressure drop from alveoli to trachea estimated at the same level or the plot 24 thereof is accordingly drawn in as a solid parallel line in relation to the straight second half 27 of the expiratory pressure-volume curve (likewise traced here in black), i.e., of the expiration process 12.

Together, the dashed parallel line and the solid parallel line reproduce quite well the (global) alveolar pressure 9 or the plot 24 of the alveolar pressure 9 during this ventilation cycle.

It is noticeable that the two parallel lines together do not form a straight line, but are slightly tilted against each other. This represents (in greatly simplified form) the central region of an S-shapedly curved dynamic compliance curve. The inflection point 31 formed by the two parallel lines correctly indicates the inflection (change in the direction of curvature). In the method described here for estimating the (global) alveolar pressure 9 or the plot 24 of the alveolar pressure 9, it is inevitably ascertained somewhat too high, especially in early inspiration, since the higher (total) resistance measured end-inspiratorily is used to this end, and not the lower end-expiratory (mainly airway-related) resistance. However, this is only relevant if a measurable tissue-related resistance builds up during the inspiration process 11, which then decreases during the expiration process 12 and (almost) disappears again at the end of the expiration process 12.

In particular, the (global) alveolar pressure 9 or the plot 24 of the alveolar pressure 9 is thus calculated on the basis of the second half 27 of the inspiration process 11 and the second half 27 of the expiration process 12, since the first fluid flow 4 from the ventilator 1 to the airway 5 and the second fluid flow 6 from the airway 5 back into the ventilator 1 or into the environment 7 are then both very stable (in terms of absolute value) and mass inertia effects, which occur especially in inhomogeneous lungs at the start of the inspiration process 11 and expiration process 12 or when changing from the inspiration process 11 to the expiration process 12 and from the expiration process 12 to the inspiration process 11, and resultant shear effects are hardly relevant.

By means of the ventilator 1 or by means of the method for differentiated measurement of the airway-related and tissue-related resistance and for ascertainment of the (global) alveolar pressure 9 or the plot 24 of the alveolar pressure 9, it is possible to ascertain and optionally output the airway-related and tissue-related resistance on the basis of the described pressure measurements according to steps c) and f) and of the respective (known) inspiratory and expiratory fluid flow 4, 6.

From the airway-related resistance and the respective (known) inspiratory and expiratory fluid flow 4, 6, it is then possible to calculate the inspiratory and expiratory pressure drop (even during ventilation) and to optionally output it (e.g., on the display of the ventilator 1). Lastly, the (global) alveolar pressure 9 or the plot 24 of the alveolar pressure 9 can be calculated and optionally output (e.g., on the display of the ventilator 1).

By means of the ventilator 1 and/or the described method, it is possible to determine and differentiate the airway-related and tissue-related resistance even over sections of (optimal, i.e., maximal) compliance especially by means of temporary stopping of the fluid flow 4, 6 during the inspiration process 11 and expiration process 12 (see, for example, FIG. 3, first ventilation cycle (far left), and FIG. 3, third ventilation cycle (second from right)).

The combination of a measurement after the set peak inspiratory pressure 25 has been reached at the first time point 13 (see, for example, FIG. 2, second ventilation cycle (second from left), with the first pressure difference 14 occurring in the subsequent time interval 16) with an interim measurement before the set end-expiratory pressure 26 has been reached at the second time point 18 (see, for example, FIG. 3, third ventilation cycle (second from the right), with the second pressure difference 19 occurring in the subsequent time interval 16) makes it possible in particular to determine and differentiate the airway-related and tissue-related resistance even more precisely and to accordingly also calculate the (global) alveolar pressure 9 or the plot 24 of the alveolar pressure 9 even more precisely.

The same is also achieved by the combination of an interim measurement at the first time point 13 before the set peak inspiratory peak 25 has been reached (see FIG. 3, first ventilation cycle (far left), with the first pressure difference 14 occurring in the subsequent time interval 16) with a measurement at the second time point 18 after the set end-expiratory pressure 26 has been reached (see, for example, FIG. 2, third ventilation cycle (second from the right), with the second pressure difference 19 occurring in the subsequent time interval 16).

Especially in the case of interim measurements, i.e., during the inspiration process 11 and before the peak inspiratory pressure 25 has been reached or during the expiration process 12 and before the end-expiratory pressure 26 has been reached, it should be borne in mind that, in the case of FCV, fluid flow conditions are (very) stable or constant especially in the second half 27 of the inspiration process 11 and in the second half 27 of the expiration process 12 and measurement conditions are therefore then particularly advantageous.

### LIST OF REFERENCE SIGNS

1 Ventilator

2 Gas supply device

3 Gas discharge device

4 First fluid flow

5 Airway

6 Second fluid flow

7 Environment

8 Pressure sensor

9 Pressure

10 Control device

11 Inspiration process

12 Expiration process

13 First time point

14 First pressure difference

15 First pressure

16 Time interval

17 Second pressure

18 Second time point

19 Second pressure difference

20 Third pressure

21 Fourth pressure

22 End-expiratory state

23 End-inspiratory state

24 Plot

25 Peak inspiratory pressure

26 End-expiratory pressure

27 Second half

28 Time

29 Volume

30 Visualization device

31 Inflection point

1. Ventilator, at least comprising a gas supply device and a gas discharge device, for supplying a first fluid flow to an airway of a patient and for discharging a second fluid flow from the airway back into the ventilator or to an environment, a pressure sensor for measuring a pressure in the airway, and a control device for operating the ventilator; wherein the fluid flow is adjustable to a constant value at least during an inspiration process and an expiration process; wherein the control device is configured to carry out a method comprising at least the following steps:
a) carrying out an inspiration process with a constant first fluid flow by means of the gas supply device,
b) stopping the first fluid flow by means of the gas supply device at a first time point, and at the same time
c) determining a first pressure difference between a first pressure present at the first time point of stopping and a second pressure occurring after a time interval by means of the pressure sensor; and
d) carrying out an expiration process with a constant second fluid flow by means of the gas discharge device,
e) stopping the second fluid flow by means of the gas discharge device at a second time point, and at the same time
f) determining a second pressure difference between a third pressure present at the second time point of stopping and a fourth pressure occurring after a time interval by means of the pressure sensor;
g) defining and providing a difference between the first pressure difference and the second pressure difference as a first index which is usable for determination of at least a tissue-related resistance of the patient.

2. Ventilator as claimed in claim 1, wherein, by carrying out steps d) to f), and when the third pressure corresponds to an end-expiratory pressure, a second index is defined and provided and an airway-related resistance of the patient is thus determinable.

3. Ventilator as claimed in claim 1, wherein it is defined for step g) that the tissue-related resistance is negligible in the end-expiratory state and maximal in the end-inspiratory state and, in between, increases linearly during the inspiration process and decreases linearly during the expiration process.

4. Ventilator as claimed in claim 3, wherein a regression analysis is performable by means of the control device at least to determine the tissue-related resistance.

5. Ventilator as claimed in claim 1, wherein the pressure sensor is arranged endotracheally.

6. Ventilator as claimed in claim 1, wherein a second index is also defined and provided by means of the control device in step g), and what are thus determinable are an airway-related resistance and, by conversion to the constant fluid flow, the pressure drop in the airway during the inspiration process and the expiration process and also an alveolar pressure or plot of an alveolar pressure.

7. Ventilator as claimed in claim 1, wherein at least the second pressure or the fourth pressure is mathematically determinable.

8. Ventilator as claimed in claim 1, wherein at least the first time point is defined in a temporal second half of the inspiration process or the second time point is defined in a temporal second half of the expiration process.

9. Ventilator as claimed in claim 1, wherein, at least in step b), the first fluid flow is stopped when a defined peak inspiratory pressure has been reached or, in step e), the second fluid flow is stopped when a defined end-expiratory pressure has been reached.

10. Ventilator as claimed in claim 9, wherein, in the case of the first pressure difference, a (total) resistance arises from a sum total of an airway-related resistance and a maximum of a tissue-related resistance.

11. Ventilator as claimed in claim 9, wherein, in the case of the second pressure difference, the (total) resistance arises from the airway-related resistance.

12. Method for determining at least a tissue-related resistance of a patient by means of a ventilator, wherein the ventilator at least a gas supply device and a gas discharge device, for supplying a first fluid flow to an airway of a patient and for discharging a second fluid flow from the airway back into the ventilator or to an environment, a pressure sensor for measuring a pressure in the airway, and a control device for operating the ventilator; wherein the fluid flow is adjustable to a constant value at least during an inspiration process and an expiration process; wherein the control device is suitably designed to carry out a method comprising at least the following steps:
a) carrying out an inspiration process with a constant first fluid flow by means of the gas supply device,
b) stopping the first fluid flow by means of the gas supply device at a first time point, and at the same time
c) determining a first pressure difference between a first pressure present at the first time point of stopping and a second pressure occurring after a time interval by means of the pressure sensor; and
d) carrying out an expiration process with a constant second fluid flow by means of the gas discharge device,
e) stopping the second fluid flow by means of the gas discharge device at a second time point, and at the same time
f) determining a second pressure difference between a third pressure present at the second time point of stopping and a fourth pressure occurring after a time interval by means of the pressure sensor;
g) defining and providing a difference between the first pressure difference and the second pressure difference as a first index and determining at least a tissue-related resistance of the patient.

13. Method as claimed in claim 12, wherein, by carrying out steps d) to f), and when the third pressure corresponds to an end-expiratory pressure, a second index is defined and provided and an airway-related resistance of the patient is thus determined.

14. Method as claimed in claim 12, wherein at least steps a) to c) during an inspiration process or steps d) to f) during an expiration process are in each case carried out multiple times together with step g).

15. Method as claimed in claim 12, wherein it is defined for step g) that the tissue-related resistance is negligible in the end-expiratory state and maximal in the end-inspiratory state and, in between, increases linearly during the inspiration process and decreases linearly during the expiration process.

16. Control device for a ventilator that is equipped, configured or programmed to carry out the method as claimed in claim 12.
